(19) United States
(12) Patent Application Publication
(10) Pub. No.: US 2008/0228102 A1
(43) Pub. Date: Sep. 18, 2008

(54) SYSTEM AND METHOD FOR PATHOLOGICAL MAPPING OF TARGETED TISSUE

(75) Inventor: Winston E. Barzell, Sarasota, FL (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: CIVCO MEDICAL INSTRUMENTS CO., INC., Kalona, IA (US)

(21) Appl. No.: 12/044,216

(22) Filed: Mar. 7, 2008

Related U.S. Application Data
(60) Provisional application No. 60/894,349, filed on Mar. 12, 2007.

(51) Int. Cl. A61B 10/02; G01N 33/48

(52) U.S. Cl. 600/562; 422/55

(57) ABSTRACT

A system and method for collecting specimens removed from different regions of targeted tissue, e.g., the prostate gland. The system is made up a plurality of templates for receipt of respective ones of the specimens and a plurality of specimen identifiers. The templates include respective areas representing corresponding to respective regions of the targeted tissue and each area bears respective visible indicia representative of the corresponding region of the targeted tissue. The specimen identifiers, e.g., label for specimen containers, include visible indicia corresponding to the respective visible indicia areas of the templates. Graphic representations of the targeted tissue showing the pathology of the regions thereof can be computer generated based on the specimens collected.

### CROSS-REFERENCE TO RELATED APPLICATIONS

This application claims priority from Provisional Application Ser. No. 60/894,349, filed on Mar. 12, 2007, entitled System and Method for Pathological Mapping of Targeted Tissue, which application is assigned to the same assignee as this application and whose disclosure is incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### BACKGROUND OF THE INVENTION

This invention relates to systems and methods for mapping targeted tissue in a living being, and more particularly to systems and methods for taking multiple core samples from multiple regions of targeted tissue within the body of a living being for pathologic analysis and for providing a graphical representation of the targeted tissue indicating its pathology.

The American Cancer Society estimates approx 220,000 new cases of prostate cancer in the United States, and 30,000 deaths attributable to the disease. Prostate cancer is suspected when a physician encounters a suspicious nodule on digital rectal exam (DRE) and/or adverse Prostate Specific Antigen (PSA) parameters (these include an elevated or rising PSA, low or falling free PSA, and an unacceptable PSA velocity). Prostate cancer is confirmed by a prostate biopsy. Currently these biopsies are performed by the transrectal route “freehand”, using a transrectal ultrasound (TRUS), and are referred to as TRUS-guided biopsies (usually 12 specimen cores are obtained). Disadvantages of the transrectal biopsy includes: a relatively poor negative predictive value, poor positional accuracy, and lack of reproducibility of sampling location. In an effort to deal with all these issues, template guided transperineal systematic biopsies have gained increasing popularity (60-80 cores are obtained). This technique has been variably referred to as template guided transperineal biopsies; transperineal saturation biopsies or mapping; and 3-D pathologic mapping (3-DPM). The technique and indications are described in my article which I co-authored with Dr. Willet F. Whitmore, entitled Transperineal Template Guided Saturation Biopsy of the Prostate: Rationale, Indications and Technique, Urology Times, Volume 31, Number 5, Pages 41-42, 2003, and in the PAACT, Inc. Prostate Cancer Communication Newsletter, Volume 19, Number 3, September 2003 which I also authored, the disclosures of both of those publications being incorporated by reference herein.

3-DPM involves obtaining multiple tissue core samples from multiple regions of the prostate using a conventional biopsy gun. These biopsies are obtained, under ultrasound guidance every 5 mm. throughout the volume of the prostate. Each specimen taken has to be transferred to some receiver on the operating room table, e.g., a piece of foam, from which it is transferred to some container or vial and sent to a pathology laboratory for histopathologic testing. Each specimen has to be appropriately labeled with data regarding the location from which it was taken and the collection vial appropriately labeled with that information. The pathology laboratory sends back a written report to the clinician indicating which of the vials contained cancerous or atypical specimens. The written report is typically several pages long in the form of narrative description of each of the specimens, e.g., if the pathologist thought that the specimen exhibited cancer, atypia or prostatic intraepithelial neoplasia (PIN). This information can then be used by the clinician to recommend and plan therapy. Therapeutic options includes watchful waiting (when little or no cancer is found), whole gland ablation by any modality (when extensive cancer is found), or focal therapy directed to the cancerous portion of the prostate only in cases where significant cancer is found in one area only. Since this mapping will form the basis for treatment recommendation and planning, it is imperative that the specimens are properly labeled and submitted.

During a typical biopsy procedure as each sample is taken and placed on some receiver, e.g., a Telfa or foam pad, the surgeon calls out (verbalizes) the region from which the specimen was taken. For example, if the surgeon removes a core of prostate tissue from the proximal portion of the midline of the gland he/she states “midline proximal” so that the person placing the specimen in/on the receiver knows where it came from. That specimen then has to be transferred to a specimen vial or jar which can be provided to pathology for analysis. To ensure that the specimen goes to the correct jar there has to be another “call-out” by the person making the transfer that the jar, in fact, bears indicia corresponding to the region from which the specimen was taken. To that end, a nurse or somebody else in the operating room that actually transfers the specimen from the receiver pad to the requisite specimen jar states something to the effect: “Transferring midline proximal into the midline proximal specimen jar.” Typically there is another nurse who holds the specimen jar and verbally repeats the identification of the specimen and the specimen jar into which it is being placed as insurance against mistake. This “call-out” procedure goes on for as many biopsies as is necessary and typically such procedures involve a relatively large number of regions of the prostate. Thus, the actions of correctly identifying/designating the specimens, placing them in appropriate containers and tagging the containers with correct data (e.g., the correct region from which the specimen was taken) in the OR during the biopsy procedure is very labor intensive, stressful and prone to human error.

Accordingly, a need exists for providing a system and method which minimizes if not eliminates the chances of human error in the identification, containerizing and tagging procedure. The subject invention addresses that need.

In addition, the methodology and system of this invention can be used to test the accuracy of MRI spectroscopy of the prostate to further develop it as a viable imaging modality. As is known MRI spectroscopy is an imaging modality for locating cancer in the prostate and basically entails a biochemical test using MRI to attempt to locate where the cancer is on a grid.

Furthermore it is extremely helpful that pathology lab's written report be supplemented with a 2-D and 3-D diagrammatic spatial representation of the areas of the prostate affected with cancer. This is necessary for proper treatment planning. The subject invention also addresses that need.

### BRIEF SUMMARY OF THE INVENTION

A system and method for collecting specimens removed from different regions of targeted tissue (e.g., the prostate gland) of a living being. The regions of the targeted tissue are located at different locations within the targeted tissue.

The system comprises a plurality of templates for receipt of respective ones of the specimens and a plurality of specimen identifiers. The plurality of templates including respective areas representing corresponding to respective regions of the targeted tissue, with each of the areas of the templates bearing respective visible indicia (e.g., color and/or alpha-numeric indicia) representative of the corresponding region of the targeted tissue. The plurality of specimen identifiers (e.g., labels for specimen containers or portions of specimen containers themselves) comprise visible indicia corresponding to the respective visible indicia areas of the templates.

The method entails removing at least one specimen from respective ones of the regions of the targeted tissue and placing the at least one specimen on the area of the template corresponding to the region from which the at least one specimen was removed. Thereafter the at least one specimen is transferred from the template on which the at least one specimen was placed into a specimen collector bearing the specimen identifier corresponding to the area of the template on which the at least one specimen had been placed.

In accordance with another aspect of the method of this invention a graphic representation of the targeted tissue (e.g., a three-dimensional graphic) is generated (e.g., a generated by computer), which graphic representation shows the pathology of the regions of the targeted tissue (e.g., the prostate gland) based on the collected specimens.

### BRIEF DESCRIPTION OF SEVERAL VIEWS OF THE DRAWINGS

The invention will be described in conjunction with the following drawings in which like reference numerals designate like elements and wherein:

FIG. 1A is an isometric view of one exemplary system for collecting specimens of targeted tissue constructed in accordance with this invention;

FIG. 1B is an enlarged isometric view of an alternative exemplary system for collecting specimens of targeted tissue constructed in accordance with this invention;

FIG. 2 is a plan view of one exemplary template of a group of templates forming a portion of the exemplary embodiment of a system shown in FIG. 1;

FIG. 2A is an enlarged plan view of a portion of the template of FIG. 2;

FIG. 3 is a plan view of another exemplary template of the exemplary group of templates constructed in accordance with this invention;

FIG. 3A is an enlarged plan view of a portion of the template of FIG. 3;

FIG. 4 is a plan view of still another exemplary template of the exemplary group of templates constructed in accordance with this invention;

FIG. 4A is an enlarged plan view of a portion of the template of FIG. 4;

FIG. 5 is a plan view of still another exemplary template of the exemplary group of templates constructed in accordance with this invention;

FIG. 5A is an enlarged plan view of a portion of the template of FIG. 5;

FIG. 6 is a plan view of still another exemplary template of the exemplary group of templates constructed in accordance with this invention;

FIG. 6A is an enlarged plan view of a portion of the template of FIG. 6;

FIG. 7 is a plan view of still another exemplary template of the exemplary group of templates constructed in accordance with this invention;

FIG. 7A is an enlarged plan view of a portion of the template of FIG. 7;

FIG. 8 is a plan view of yet another exemplary template of the exemplary group of templates constructed in accordance with this invention;

FIG. 8A is an enlarged plan view of a portion of the template of FIG. 8;

FIG. 9A is a block diagram of one exemplary method of the subject invention;

FIG. 9B is a block diagram, similar to FIG. 9A, but showing an alternative method of the subject invention;

FIG. 10 is a plan view of a set of labels for collection jars or vials forming a portion of the exemplary system shown in FIG. 1;

FIG. 11A is an exploded isometric illustration of the prostate gland showing the various regions from which specimens can be collected utilizing the system and method of the subject invention;

FIG. 11B is a top plan view of the illustration of FIG. 11A taken along line 11B-11B; and

FIG. 11C is a bottom plan view of the illustration of FIG. 11A taken along line 11C-11C.

### DETAILED DESCRIPTION OF THE PREFERRED EMBODIMENTS OF THE INVENTION

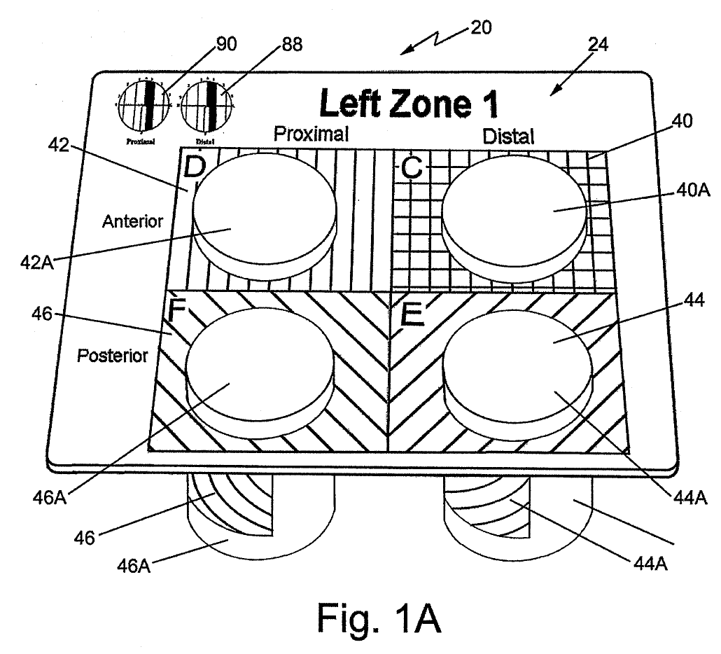

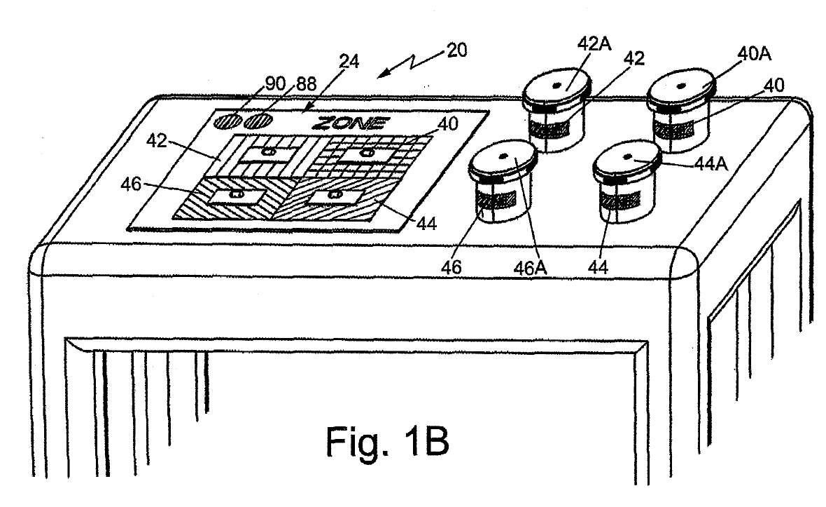

Referring now to the drawing wherein like characters refer to like parts, there is shown in FIG. 1A a system 20 for collecting specimens (cores) of any targeted tissue within the body of a being. In the exemplary embodiment shown the targeted tissue comprises the prostate gland. The system basically comprises a plurality of templates, each of which includes plural specimen receiving vials or jars and a plurality of labels for the vials/jars. In particular, the plurality of labels are arranged to be used on respective ones of a plurality of specimen vials/jars. The vials or jars preferably form portions of the templates and are releasably disposed therein, although they may be permanently part of the templates. Alternatively, the vials or jars may be separate items and the templates separate items as shown in the embodiment of FIG. 1B.

The details of the templates, labels and vials/jars will be described later. Suffice it for now to state that in accordance with the exemplary embodiment of the system shown in FIG. 1A, each of the templates is a generally tray-shaped member that includes plural colored areas, each of which is arranged to receive one or more specimens of the targeted tissue removed from the being to enable the targeted tissue to be mapped. To that end each of the colored areas holds a respective specimen collection vial or jar. Each jar includes a respectively colored label on it. The colored areas of the template represent respective ones of various respective regions of the targeted tissue from which the specimens are to be taken. Each colored area of the template also includes other indicia, namely, an alpha-numeric character (e.g., a letters of the alphabet) representing the respective region of the targeted tissue. In addition, the template also includes other alpha-numeric indicia, e.g., words describing the region(s) indicated by the template.

The mapping of the prostate 10 is accomplished by assigning and figuratively breaking up the prostate into various zones or regions and taking at least one biopsy specimen or core from in each of those regions. In particular, at least one specimen (core) 12 is removed from each region and labeled in accordance with the Cartesian coordinates as to where it came from, e.g., each core is obtained from a specific coordinate on the X and Y axis, and is labeled proximal or distal depending on its location on the Z axis. These cores are either submitted separately, or in various groups depending on the indication for the 3-D mapping, and on surgeon preference.

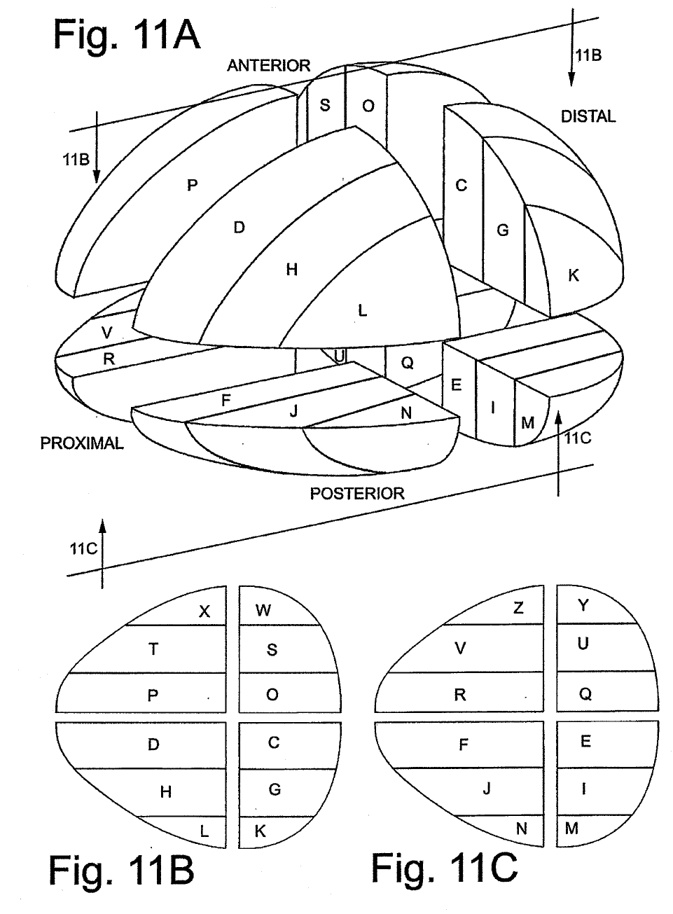

In accordance with one preferred exemplary embodiment of this invention, and as best seen in the illustrations of FIG. 11A-11C, the prostate gland 10 is broken down into twenty-six regions for analysis. In the interest of drawing simplicity two of those regions, namely, the Midline Distal region and the Midline Proximal region are not shown. The remaining regions are Left Zone 1 Distal Anterior region C, Left Zone 1 Proximal Anterior region D, Left Zone 1 Distal Posterior region E, Left Zone 1 Proximal Posterior region F, Left Zone 2 Distal Anterior region G, Left Zone 2 Proximal Anterior region H, Left Zone 2 Distal Posterior region I, Left Zone 2 Proximal Posterior region J, Left Zone 3 Distal Anterior region K, Left Zone 3 Proximal Anterior region L, Left Zone 3 Distal Posterior region M, Left Zone 3 Proximal Posterior region N, Right Zone 1 Distal Anterior region O, Right Zone 1 Proximal Anterior region P, Right Zone 1 Distal Posterior region Q, Right Zone I Proximal Posterior region R, Right Zone 2 Distal Anterior region S, Right Zone 2 Proximal Anterior region T, Right Zone 2 Distal Posterior region U, Right Zone 2 Proximal Posterior region V, Right Zone 3 Distal Anterior region W, Right Zone 3 Proximal Anterior region X, Right Zone 3 Distal Posterior region Y and Right Zone 3 Proximal Posterior region Z.

Accordingly, for applications involving taking biopsy specimens from those regions the system 20 includes seven templates 22, 24, 26, 28, 30, 32 and 34 (to be described later) and twenty-six labels 36, 38, 40, 42, 44, 46, 48, 50, 52, 54, 56, 58, 60, 62, 64, 66, 68, 70, 72, 74, 76, 78, 80, 82, 84 and 86 (also to be described later). The labels are used on the twenty-six specimen collection vials or jars 36A, 38A, 40A, 42A, 44A, 46A, 48A, 50A, 52A, 54A, 56A, 58A, 60A, 62A, 64A, 66A, 68A, 70A, 72A, 74A, 76A, 78A, 80A, 82A, 84A and 86A (also to be described later). As mentioned earlier the vial or jars preferably form parts of the templates or may be separate components.

It should be pointed out at this juncture that the particular number of regions of the targeted tissue to be examined is purely a function of the amount of information needed for a particular procedure. For example, mapping of the prostate for seeding or brachiotherapy or removal of just the cancerous region of the prostate needn't assay as many regions as for scientific research. As mentioned earlier the exemplary embodiment described herein is arranged to sample twenty-six regions of the prostate, with at least one specimen taken from each of those regions. That is merely exemplary. Thus, for some application only eight regions or octants of the prostate may be sampled.

If the templates include the vials or jars, like in the embodiment of FIG. 1A, the specimen(s) taken are introduced from the biopsy gun directly into the appropriate jar in accordance with the color coding established by the templates. If the jars are separate from the templates, then the specimens taken are first placed on the appropriate colored areas of the templates and then transferred to the corresponding vials or jars.

Each template of the embodiment of FIG. 1A basically comprises a generally planar tray shaped member having plural holes in it. Each hole is adapted to releasably receive a respective specimen jar therein. The portion of the tray contiguous with the specimen jar is colored and bears alpha-numeric indicia indicating the region of the prostate from which the specimens for that jar have been taken.

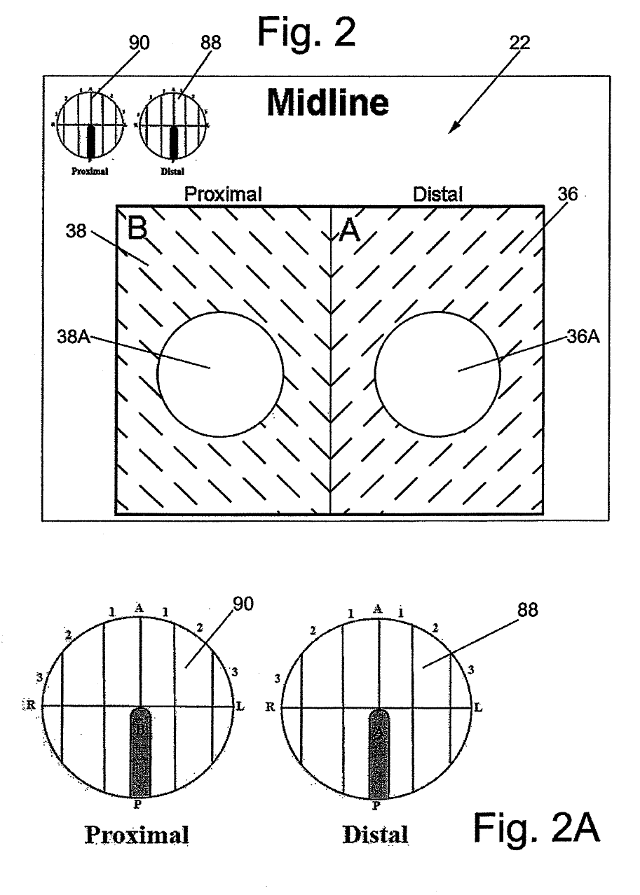

Referring now to FIG. 2, the details of the template 22 will now be described. That template identifies what is referred to as the “Midline” zone of the prostate and includes two regions of that zone, namely the “Distal” region “A” and the “Proximal” region “B”, each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the midline distal area of the template bears the indicium “A” and is colored green, while the midline proximal area bears the indicium “B” and is colored pink. It should be pointed out at this juncture that the particular colors used in this template and in any other template to be described later are merely exemplary. Thus, any colors can be used, provided that each region has its own respective color which is different than other colors of regions of the associated zone to enable one to readily distinguish the regions of any particular zone from one another.

As mentioned earlier, each of the colored areas of each template is arranged to releasably receive a respective specimen collecting vial or jar. To that end, each area includes the heretofore identified hole, into which a corresponding specimen collection vial or jar is placed. In addition, the template 22 includes two orientation guide diagrams 88 and 90. Each orientation guide diagram is taken along a sagittal plane of the prostate gland. Diagram 88 shows the location of the Midline Distal region A, while diagram 90 shows the location of the Midline Proximal region B. The diagrams 88 and 90 are shown enlarged in FIG. 2A. If the template is constructed so that it will serve as a temporary receiver for the specimens collected, i.e., the specimens will be temporarily transferred to it and then transferred to respective collection vials/jars, the colored areas of the templates may include respective Telfa or foam pads 14, as shown in FIG. 1B.

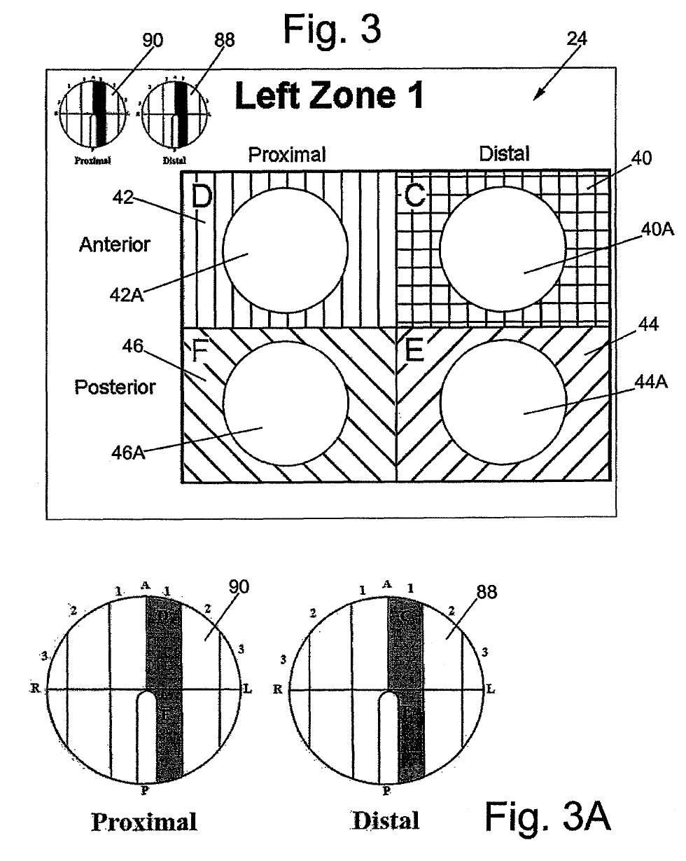

The template 24 is shown in FIGS. 3 and 3A and identifies what is referred to as the “Left Zone 1” that includes four regions of that zone, namely the “Distal Anterior” region “C,” the “Proximal Anterior” region “D”, the “Distal Posterior” region “E” and the “Proximal Posterior” region “F”, each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue and bears the indicium “C”, the proximal anterior area is colored yellow and bears the indicium “D”, the distal posterior area is colored white and bears the indicium “E” and the proximal posterior area is colored red and bears the indicium “F.” Each of the areas C-F is also arranged to receive a respective biopsy specimen and thus includes a portion of foam. Like template 22, template 24 includes two orientation guide diagrams 88 and 90. Diagram 88 shows the location of the Left Zone 1 distal regions C and E, while diagram 90 shows the location of the Left Zone 1 proximal regions D and F. The diagrams 88 and 90 are shown enlarged in FIG. 3A.

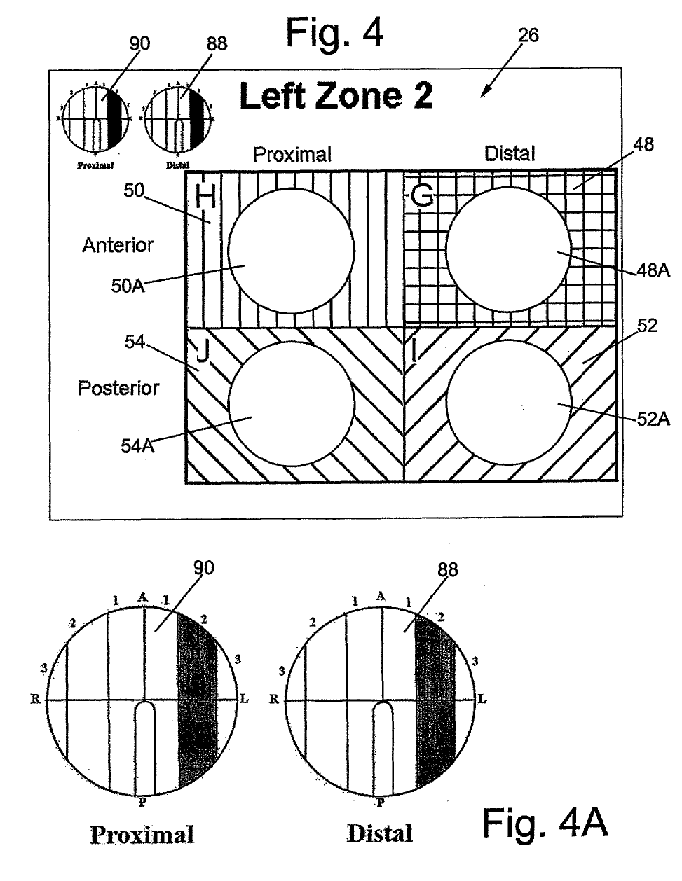

The template 26 is shown in FIGS. 4 and 4A and identifies what is referred to as the “Left Zone 2” that includes four regions of that zone, namely the “Distal Anterior” region “G,” the “Proximal Anterior” region “H”, the “Distal Posterior” region “T” and the “Proximal Posterior” region “J”, each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue and bears the indicium “G”, the proximal anterior area is colored yellow and bears the indicium “H”, the distal posterior area is colored white bears the indicium “I” and the proximal posterior area is colored red and bears the indicium “J.” Each of the areas G-I is also arranged to receive a respective biopsy specimen and thus includes a portion of foam. Template 26 also includes two orientation guide diagrams 88 and 90. Diagram 88 shows the location of the Left Zone 2 distal regions G and I, while diagram 90 shows the location of the Left Zone 2 proximal regions H and J. The diagrams 88 and 90 are shown enlarged in FIG. 4A.

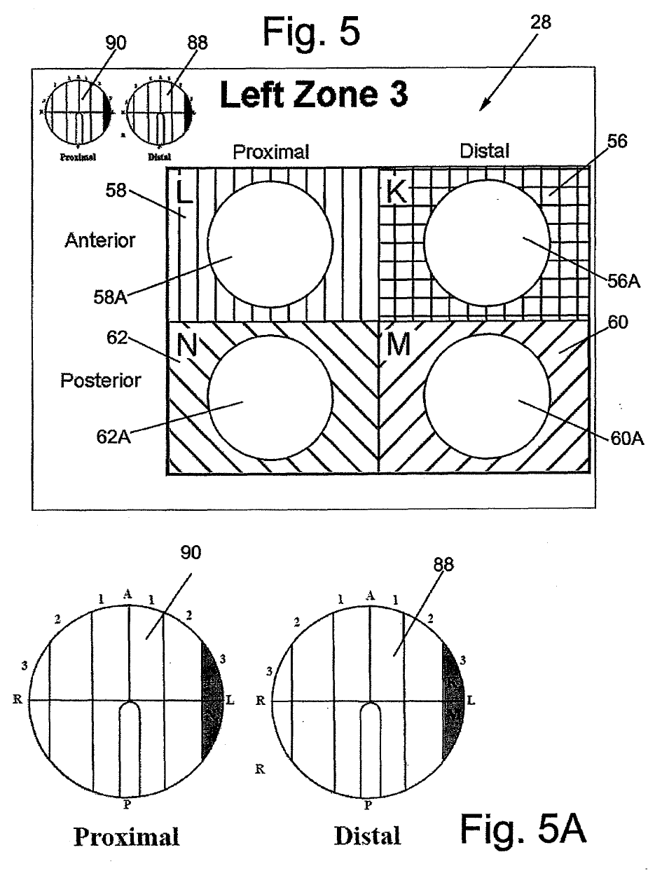

The template 28 is shown in FIGS. 5 and 5A and identifies what is referred to as the “Left Zone 3” that includes four regions of that zone, namely the “Distal Anterior” region “K,” the “Proximal Anterior” region “L”, the “Distal Posterior” region “M” and the “Proximal Posterior” region “N” each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue and bears the indicium “K”, the proximal anterior area is colored yellow and bears the indicium “L”, the distal posterior area is colored white and bears the indicium “M” and the proximal posterior area is colored red and bears the indicium “N.” Each of the areas K-N is also arranged to receive a respective biopsy specimen and thus includes a portion of foam. Template 28 also includes two orientation guide diagrams 88 and 90. Diagram 88 shows the location of the Left Zone 3 distal regions K and M, while diagram 90 shows the location of the Left Zone 3 proximal regions L and N. The diagrams 88 and 90 are shown enlarged in FIG. 5A.

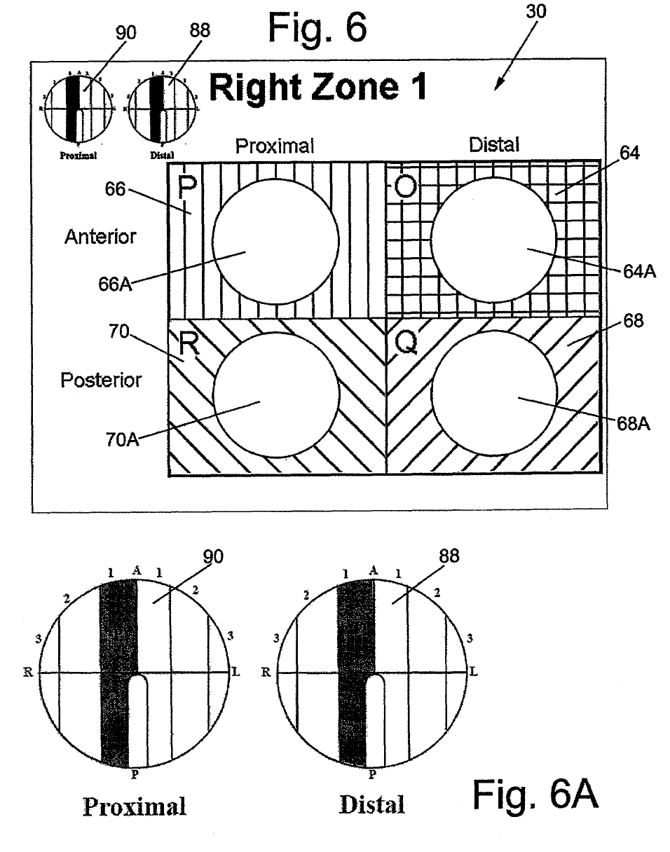

The template 30 shown in FIGS. 6 and 6A and identifies what is referred to as the “Right Zone 1” that includes four regions of that zone, namely the “Distal Anterior” region “O” the “Proximal Anterior” region “P”, the “Distal Posterior” region “Q” and the “Proximal Posterior” region “R” each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue bears the indicium “O”, the proximal anterior area is colored yellow and bears the indicium “P”, the distal posterior area is colored white and bears the indicium “Q” and the proximal posterior area is colored red and bears the indicium “R.” Each of the areas O-R is also arranged to receive a respective biopsy specimen and thus includes a portion of foam. Template 30 also includes two orientation guide diagrams 88 and 90. Diagram 88 shows the location of the Right Zone 1 distal regions O and Q, while diagram 90 shows the location of the Right Zone 1 proximal regions P and R. The diagrams 88 and 90 are shown enlarged in FIG. 6A.

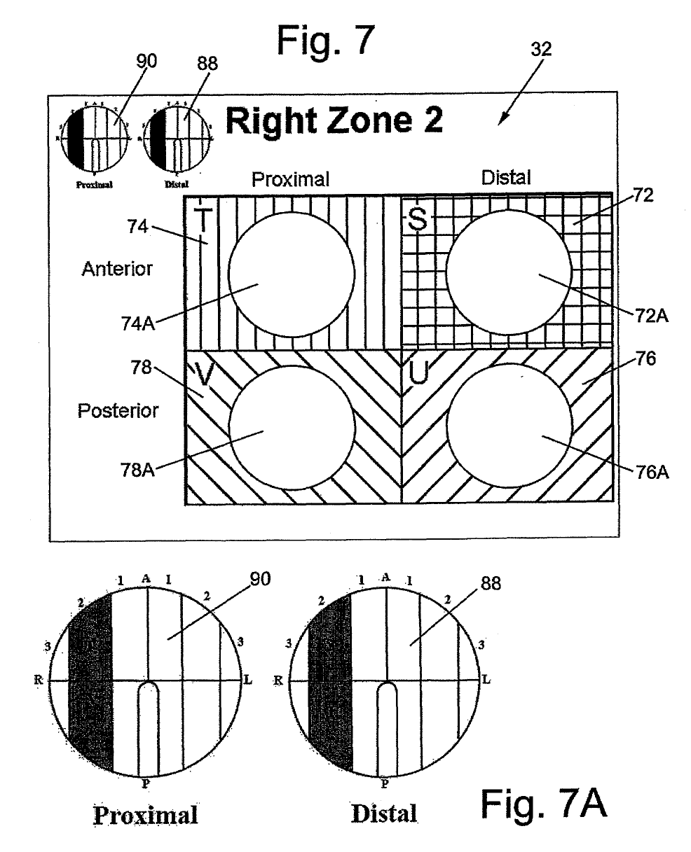

The template 32 is shown in FIGS. 7 and 7A and identifies what is referred to as the “Right Zone 2” that includes four regions of that zone, namely the “Distal Anterior” region “S” the “Proximal Anterior” region “T”, the “Distal Posterior” region “U” and the “Proximal Posterior” region “V” each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue and bears the indicium “S”, the proximal anterior area is colored yellow and bears the indicium “T”, the distal posterior area is colored white and bears the indicium “U” and the proximal posterior area is colored red and bears the indicium “V.” Each of the areas S-V is also arranged to receive a respective biopsy specimen and thus includes a portion of foam. Template 32 also includes two orientation guide diagrams 88 and 90. Diagram 88 shows the location of the Right Zone 2 distal regions S and U, while diagram 90 shows the location of the Right Zone 2 proximal regions T and V. The diagrams 88 and 90 are shown enlarged in FIG. 7A.

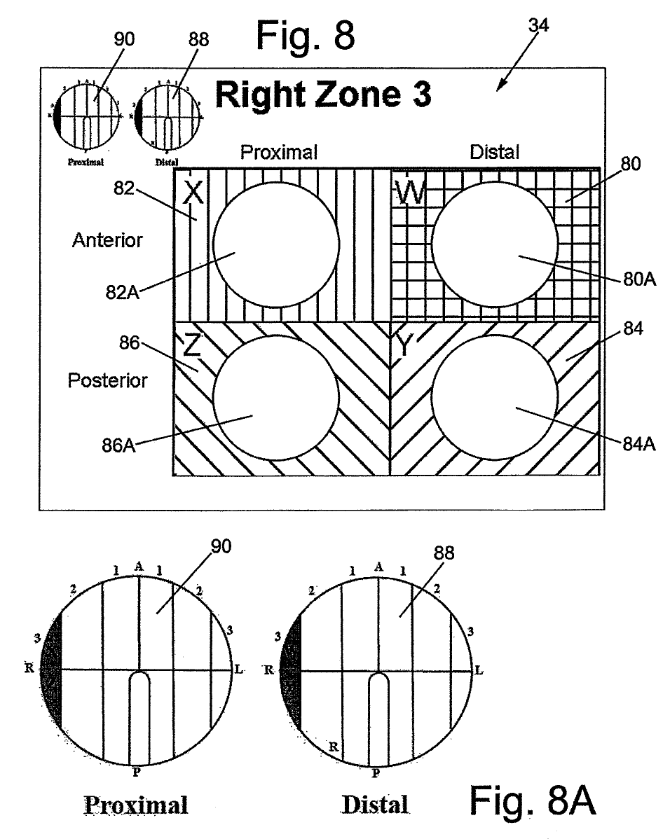

The template 34 is shown in FIGS. 8 and 8A and identifies what is referred to as the “Right Zone 3” that includes four regions of that zone, namely the “Distal Anterior” region “W” the “Proximal Anterior” region “X”, the “Distal Posterior” region “Y” and the “Proximal Posterior” region “Z” each of which is shown by a rectangle of a respective color (the respective colors being designated by the respective hatch lines in that figure). Each region is in the form of a rectangular area that not only bears its respective color indicium, but also includes a respective alpha-numeric indicium. Thus, the distal anterior area of the template is colored blue and bears the indicium “W”, the proximal anterior area is colored yellow and bears the indicium “X”, the distal posterior area is colored white and bears the indicium “T” and the proximal posterior area is colored red and bears the indicium “Z.” Each of the areas W-Z is also arranged to receive a respective biopsy specimen and thus includes a portion of foam. Template 34 also includes two orientation guide diagrams 88 and 90. Diagram 88 shows the location of the Right Zone 3 distal regions W and Y, while diagram 90 shows the location of the Right Zone 3 proximal regions X and Z. The diagrams 88 and 90 are shown enlarged in FIG. 8A.

With the subject invention when the surgeon is about to take specimens from a particular region of the prostate, such as the Right Zone 1 region, the template 30 for that region is utilized. As the surgeon takes the specimen from that zone's four possible regions, all he has to do is to call out the color of the particular region from which the specimen is being taken so that that particular specimen 12 can be placed on the appropriately colored area of the template. For example, if the specimen is being taken from the Right Zone 1 Proximal Posterior region R all that the surgeon has to say is “red,” instead of calling out the words describing that region. The person making the transfer from the biopsy gun to the template merely has to dispose that biopsy specimen 12 on the red colored area of the template, thereby reducing the change of error. This can be accomplished while the surgeon is using a second biopsy gun to take another specimen. Once that other specimen has been taken the surgeon merely has to call out the color representing the region from which the other specimen has been taken so that that specimen can be placed on the correspondingly colored area of the template.

In accordance with one exemplary embodiment of the system of this invention, the relevant template for the particular zone and its regions from which specimens will be taken is arranged to releasably hold plural vials or jars for receipt of the specimens, with those vials or jars being located in respectively colored areas of the template. Moreover, those specimen jars bear appropriate indicia (e.g., color and alpha-numeric) to indicate the zone and region from which the specimens were taken. To that end the relevant template for the particular zone and regions include respective holes in each of its respective colored areas, with a respective bottle or vial being releasably disposable in each of those holes. For example, as can be seen in FIGS. 1A and 3, the red area F of template 24 designating Left Zone 1 Proximal Posterior includes a bottle or vial 46A for receipt of each specimen 12 taken from that region. Moreover, that bottle or jar also bears a label 46 of the same color and alpha-numeric indicia indicating the respective region, e.g., a red label 46 bearing the indicia Left Zone 1 Proximal Posterior F thereon. The label may include other indicia, e.g., the name of the surgeon, patient, date of the procedure, etc.

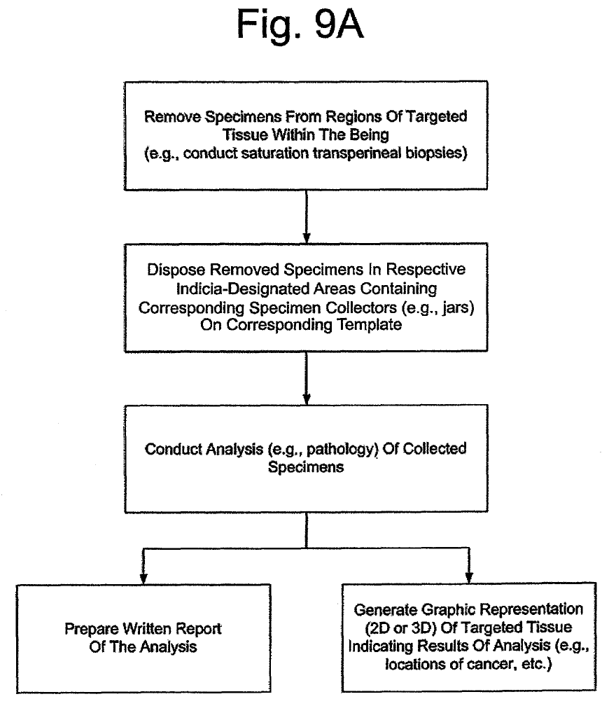

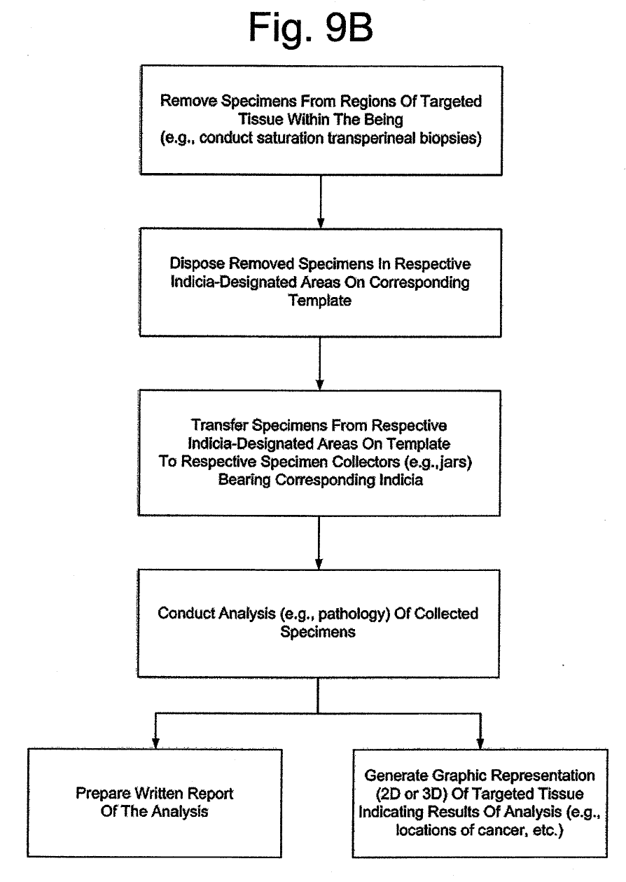

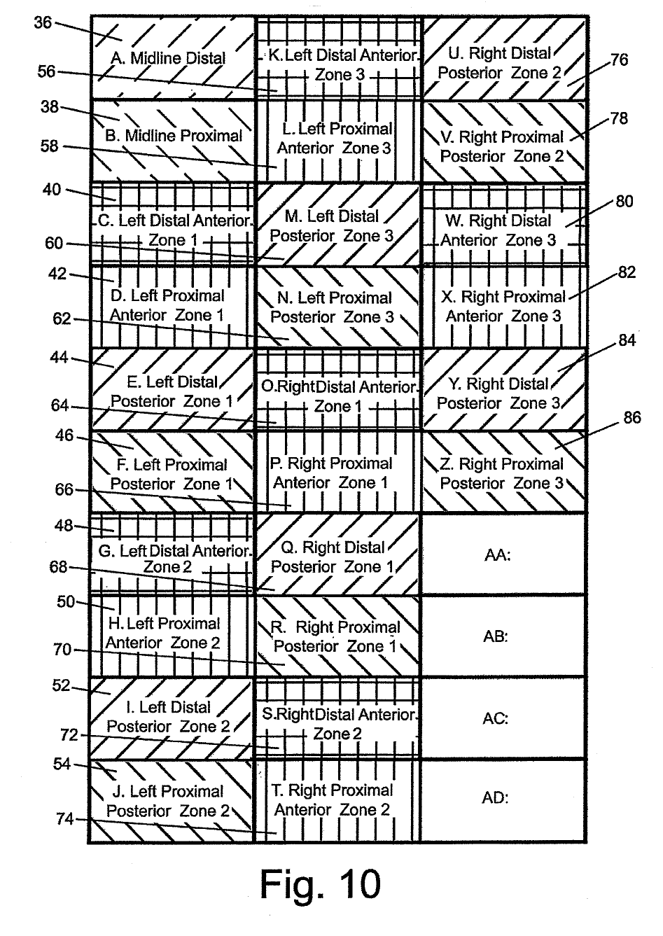

One method for using the system 20 as shown and described with reference to FIG. 1A is shown in the block diagram of FIG. 9A, with the steps thereof being described in the boxes of that figure.

If the system 20 is configured so that its templates do not include the specimen receiving vials or jars like shown in FIG. 1A, such as if the templates are merely used for temporary storage of the specimens until they are transferred to the specimen vials or jars, the colored areas of the templates may include a pad 14, such as Telfa or foam, for direct receipt of the specimen(s) thereon as shown in FIG. 1B. The procedure for placing the collected specimens in their jars is slightly different than the procedure described above. In such a case, and using the same example as above, i.e., removal of a specimen from the Left Zone 1 Proximal Posterior region, the specimen 12 when removed from that region is placed on the foam or Telfa pad of in the red area 46 of the template 24. At some later time, e.g., when the specimen(s) 12 for the other regions of that template have been taken and placed on their appropriately colored areas of the template 24, the template can be carried over to the location of the specimen receiving vials or jars. All that is necessary is to pick the specimen(s) 12 from the red area F (46) of the template 24 and place it in the jar 46A bearing the red label 46 (which label also bears the indicia Left Zone 1 Proximal Posterior F). One method for carrying out the foregoing procedure is shown in the block diagram of FIG. 9B, with the steps thereof being described in the boxes of that figure.

It should be pointed out at this juncture that once the specimens have been analyzed the findings can be provided to a computer system (not shown) for generating either a 2-dimensional (“2-D”) or a 3-dimensional (“3-D”) view of the targeted tissue, e.g., the prostate, showing areas of abnormal pathology. The generation of a 3-D view is preferred since it is most representative of actually examining the target tissue in one's hands, particularly if the 3-D view can be rotated in space by appropriate computer software.

As should be appreciated from the foregoing the system and method of this invention enables the collection, organization, labeling, and pathology submission necessary for appropriate mapping of any targeted tissue, while minimizing the chances of human error. In addition, the data representative of the pathology of the various specimens taken from the various regions of the targeted tissue as typically described in a conventional written pathology report can be supplemented with a 2-D and 3-D diagrammatic spatial representation of the areas having abnormal or atypical pathology, e.g., cancer, or pre-cancerous conditions, such as atypia or PIN. This can be accomplished by the manual method of specimen organization as described above, or by a fully automated computerized system. Such a system may make use an electronic stepper, and real time 3-D rendering of the exact shape of the prostate, and a real time capture of precise area being biopsied within prostate, with a subsequent rendition (after pathologic examination) of the prostate with area(s) afflicted with cancer, atypia and PIN.

While the invention has been described in detail and with reference to specific embodiments thereof, it will be apparent to one skilled in the art that various changes and modifications can be made therein without departing from the spirit and scope thereof.

What is claimed is:

1. A system for collecting specimens removed from different regions of targeted tissue of a living being, the regions being located at different locations within the targeted tissue, said system comprising a plurality of templates for receipt of respective ones of the specimens and a plurality of specimen identifiers, said plurality of templates including respective areas representing corresponding to respective regions of the targeted tissue, each of said areas of said templates bearing respective visible indicia representative of the corresponding region of the targeted tissue, said plurality of specimen identifiers comprising visible indicia corresponding to the respective visible indicia areas of said templates.

2. The system of claim 1 wherein said respective visible indicia comprise color and/or alpha-numeric indicia.

3. The system of claim 1 wherein said specimen identifiers are arranged to be coupled to or to form a portion of specimen container for at least one specimen.

4. The system of claim 1 wherein said specimen identifiers comprise labels for use on respective specimen containers.

5. A method for collecting specimens removed from plural regions of targeted tissue of a living being, said regions being located at different locations within said targeted tissue, said method comprising:
a) providing a plurality of templates for receipt of respective ones of the specimens, said plurality of templates including respective areas representing corresponding respective regions of the targeted tissue, each of said areas of said templates bearing respective visible indicia representative of the corresponding three dimensional region of the targeted tissue,
b) providing a plurality of specimen identifiers, said plurality of specimen identifiers comprising visible indicia corresponding to the respective visible indicia areas of said templates and being arranged to form part of or be coupled to respective specimen collectors,
c) removing at least one specimen from respective ones of said regions of the targeted tissue and placing said at least one specimen on the area of said template corresponding to the region from which said at least one specimen was removed,
d) transferring said at least one specimen from the template on which said at least one specimen was placed into a specimen collector bearing the specimen identifier corresponding to the area of said template on which said at least one specimen had been placed.

6. The method of claim 5 wherein said respective visible indicia comprise color and/or alpha-numeric indicia.

7. The method of claim 5 wherein the targeted tissue comprises the prostate gland and wherein the specimens are collected by transperineal saturation biopsy.

8. The method of claim 7 wherein said regions of the prostate gland comprise the left anterior distal portion, the left anterior proximal portion, the left posterior distal portion, the left anterior proximal portion, the right anterior distal portion, the right anterior proximal portion, the right posterior distal portion, and the right anterior proximal portion.

9. The method of claim 7 wherein said regions of the prostate gland comprise three respective zones of the left anterior distal portion, three respective zones of the left anterior proximal portion, three respective zones of the left posterior distal portion, three respective zones of the left anterior proximal portion, three respective zones of the right anterior distal portion, three respective zones of the right anterior proximal portion, three respective zones of the right posterior distal portion, and three respective zones of the right anterior proximal portion.

10. The method of claim 5 wherein said method additionally comprises generating a graphic representation of the targeted tissue showing the pathology of said regions based on said collected specimens.

11. The method of claim 10 wherein said graphic representation is three-dimensional.

12. The method of claim 10 wherein said graphic representation is computer-generated.

13. The method of claim 10 wherein the targeted tissue comprises the prostate gland and wherein the specimens are collected by transperineal saturation biopsy.

14. The method of claim 13 wherein said regions of the prostate gland comprise the left anterior distal portion, the left anterior proximal portion, the left posterior distal portion, the left anterior proximal portion, the right anterior distal portion, the right anterior proximal portion, the right posterior distal portion, and the right anterior proximal portion.

15. The method of claim 13 wherein said regions of the prostate gland comprise three respective zones of the left anterior distal portion, three respective zones of the left anterior proximal portion, three respective zones of the left posterior distal portion, three respective zones of the left anterior proximal portion, three respective zones of the right anterior distal portion, three respective zones of the right anterior proximal portion, three respective zones of the right posterior distal portion, and three respective zones of the right anterior proximal portion.

16. The method of claim 14 wherein said graphic representation is three-dimensional.

17. The method of claim 16 wherein said graphic representation is computer-generated.
